(19) United States
(12) Patent Application Publication
(10) Pub. No.: US 2023/0284980 A1
(43) Pub. Date: Sep. 14, 2023

(54) DETECTING POSITION OF A WEARABLE MONITOR

(71) Applicant: Whoop, Inc., Boston, MA (US)

(72) Inventors: Eric Alexander Otte, Boston, MA (US); Mostafa Ghannad-Rezaie, Malden, MA (US); Behnoosh Tavakoli, Needham, MA (US); Daniel Philip Wiese, Washington, DC (US)

(21) Appl. No.: 18/182,197

(22) Filed: Mar. 10, 2023

Related U.S. Application Data
(60) Provisional application No. 63/319,038, filed on Mar. 11, 2022.

(51) Int. Cl. A61B 5/00; A61B 5/024

(52) U.S. Cl. CPC A61B 5/684; A61B 5/02416

(57) ABSTRACT

A physiological monitor uses a light source and a number of detectors to determine whether a physiological monitor is positioned for acquisition of physiological data. More specifically, an intensity of the light source, as measured at two photodetectors at different distances from the light source, can be used to accurately detect whether the monitor is properly positioned for use. The disclosed methods may advantageously leverage existing physiological monitoring hardware (such as light emitting diodes and photodetectors), and may improve on the accuracy of prior art techniques using, e.g., capacitive sensors and/or other hardware to detect proper device positioning.

### CROSS-REFERENCE TO RELATED APPLICATIONS

This application claims priority to U.S. Provisional Patent App. No. 63/319,038 filed on Mar. 11, 2022, the entire contents of which are hereby incorporated by reference herein.

### TECHNICAL FIELD

This disclosure generally relates to physiological monitoring systems, and more specifically to techniques for detecting whether a physiological monitoring system is properly positioned for acquisition of data.

### BACKGROUND

Wearable physiological monitors can provide a wealth of physiological data from a wearer. There remains a need for improved methods and systems for determining whether a physiological monitor is positioned for acquisition of physiological data.

### SUMMARY

A physiological monitor uses a light source and a number of detectors to determine whether a physiological monitor is positioned for acquisition of physiological data. More specifically, an intensity of the light source, as measured at two photodetectors at different distances from the light source, can be used to accurately detect whether the monitor is properly positioned for use. The disclosed methods may advantageously leverage existing physiological monitoring hardware (such as light emitting diodes and photodetectors), and may improve on the accuracy of prior art techniques using, e.g., capacitive sensors and/or other hardware to detect proper device positioning.

In an aspect, a wearable physiological monitor disclosed herein may include: a strap configured to retain the wearable physiological monitor in a position for use on a user; a motion sensor; an illumination source directed toward a skin of a user when the wearable physiological monitor is retained in the position for use by the strap; a first optical sensor positioned a first distance from the illumination source and oriented to detect a first intensity of light from the illumination source reflected from the skin of the user; a second optical sensor positioned a second distance from the illumination source greater than the first distance, the second optical sensor oriented to detect a second intensity of light from the illumination source reflected from the skin of the user; a light emitting diode positioned to provide a visual indicator to the user when the wearable physiological monitor is retained in the position for use by the strap; and a processor. The processor may be configured by computer executable code stored in a memory to perform the steps of: detecting, with a signal from the motion sensor, a transition event indicative of placing the wearable physiological monitor for use on the user; in response to detecting the transition event, providing illumination from the illumination source; measuring the first intensity of the illumination with the first optical sensor; measuring the second intensity of the illumination with the second optical sensor; determining whether the wearable physiological monitor is positioned for use on skin of the user based on a comparison of the first intensity to the second intensity; and illuminating the visual indicator in response to determining that the wearable physiological monitor is positioned for use.

In an aspect, a computer program product disclosed herein may include computer executable code embodied in a non-transitory computer readable medium that, when executing on one or more processors of a wearable device, causes the wearable device to perform the step of detecting, with a motion sensor of the wearable device, a transition event indicative of placing the monitor for use on a user. In response to detecting the transition event, the following steps may be performed: providing illumination from a light emitting diode of the wearable device; measuring a first intensity of the illumination with a first optical sensor a first distance from the light emitting diode; measuring a second intensity of the illumination with a second optical sensor a second distance from the light emitting diode, where the second distance from the light emitting diode is greater than the first distance from the light emitting diode; and determining whether the wearable device is positioned for use on a skin of the user based on a comparison of the first intensity to the second intensity. The computer program product may also include code that, when executed, causes the wearable device to perform the step of displaying an indicator to the user of whether the wearable device is positioned for use with a light emitting diode on the wearable device. Displaying the indicator may include illuminating the light emitting diode when the wearable device is properly positioned for use.

In an aspect, a method disclosed herein may include detecting a transition event indicative of placing a wearable monitor for use on a user. In response to detecting the transition event, the following steps may be performed: providing illumination from a light emitting diode of the wearable monitor; measuring a first intensity of the illumination with a first optical sensor a first distance from the light emitting diode; measuring a second intensity of the illumination with a second optical sensor a second distance from the light emitting diode, where the second distance from the light emitting diode is greater than the first distance from the light emitting diode; and determining whether the wearable monitor is positioned for use on a skin of the user for acquisition of physiological data based on a comparison of the first intensity to the second intensity. The method may also include displaying an indicator to the user of whether the wearable monitor is positioned for use.

In an aspect, a device disclosed herein may include: a physiological monitor including a light source providing a source of illumination, a first sensor responsive to the illumination and positioned a first distance from the light source, and a second sensor responsive to the illumination from the light source and positioned at a second distance greater than the first distance from the light source; and a processor within the physiological monitor, the processor configured to activate the light source to provide the illumination, to measure a first intensity of the illumination at the first sensor, to measure a second intensity of the illumination at the second sensor, and to determine whether the physiological monitor is positioned for use on a skin of a user based on a comparison of the first intensity to the second intensity.

Implementations may include one or more of the following features. The device may further include an accelerometer, where the processor is configured to activate the light source in response to motion detected by the accelerometer. The device may further include a capacitive touch sensor, where the processor is configured to activate the light source in response to motion detected by the capacitive touch sensor. The processor may be configured to activate the light source and measure illumination in response to detecting a motion of the physiological monitor indicative of placement for use. The comparison may be a function of the first intensity and the second intensity. The comparison may be based on a ratio of the first intensity to the second intensity. The comparison may be based on a range between the first intensity and the second intensity. The comparison may be performed by a state machine, the state machine including at least one state transition based on movement detected with an accelerometer of the physiological monitor. The comparison may be performed by a state machine, the state machine including at least one state transition based on a capacitive sensor of the physiological monitor. The physiological monitor may include a heart rate monitor. The physiological monitor may include a photoplethysmography system. The processor may be configured to, in response to determining that the physiological monitor is positioned for use on the skin of the user, perform a physiological monitoring process with the physiological monitor. The physiological monitoring process may include one or more of heart rate monitoring and peripheral oxygen saturation monitoring. The processor may be configured to, in response to detecting a transition from positioned for use to not positioned for use, stop a physiological monitoring process by the physiological monitor. At least one of the first intensity and the second intensity may be measured in an infrared range. The light source may include an infrared light emitting diode. The first intensity may be measured as an average over an interval. The interval may be at least three seconds. The interval may be about five seconds. The second intensity may be measured as an average over an interval. The interval may be at least three seconds. The interval may be about five seconds. The comparison may be performed by a machine learning algorithm trained with training data that is labeled as on wrist or off wrist. The comparison may be performed by a machine learning algorithm trained with data labeled as on skin or off skin. The training data may include accelerometer data. The training data may include ambient light data. The training data may include capacitive touch sensor data. The training data may include one or more features of the first intensity and the second intensity. The device may be configured to, in response to determining that the physiological monitor is positioned for use on a skin of a user, provide a notification to the user that the device is ready for use.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1 shows a physiological monitoring device.

FIG. 2 illustrates a physiological monitoring system.

FIG. 3 shows a sensing system.

FIG. 4 shows a flow chart of a method for detecting device position.

FIG. 5 shows a state machine implementing an on/off detection process.

### DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “user” as used herein, refers to any type of animal, human or non-human, whose physiological information may be monitored using an exemplary wearable physiological monitoring system.

The term “continuous,” as used herein in connection with heart rate data collection, refers to collection of heart rate data at a sufficient frequency to enable detection of every heartbeat and also refers to collection of heart rate data continuously throughout the day and night.

The term “computer-readable medium,” as used herein, refers to a non-transitory storage hardware, non-transitory storage device or non-transitory computer system memory that may be accessed by a controller, a microcontroller, a microprocessor, a computational system, or a module of a computational system to encode thereon computer-executable instructions or software programs. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), computer system memory or random access memory (such as, DRAM, SRAM, EDO RAM) and the like.

U.S. Pat. No. 11,185,292 describes non-limiting example embodiments of physiological monitoring systems on which the systems and methods described herein may be deployed, and is incorporated by reference herein in its entirety.

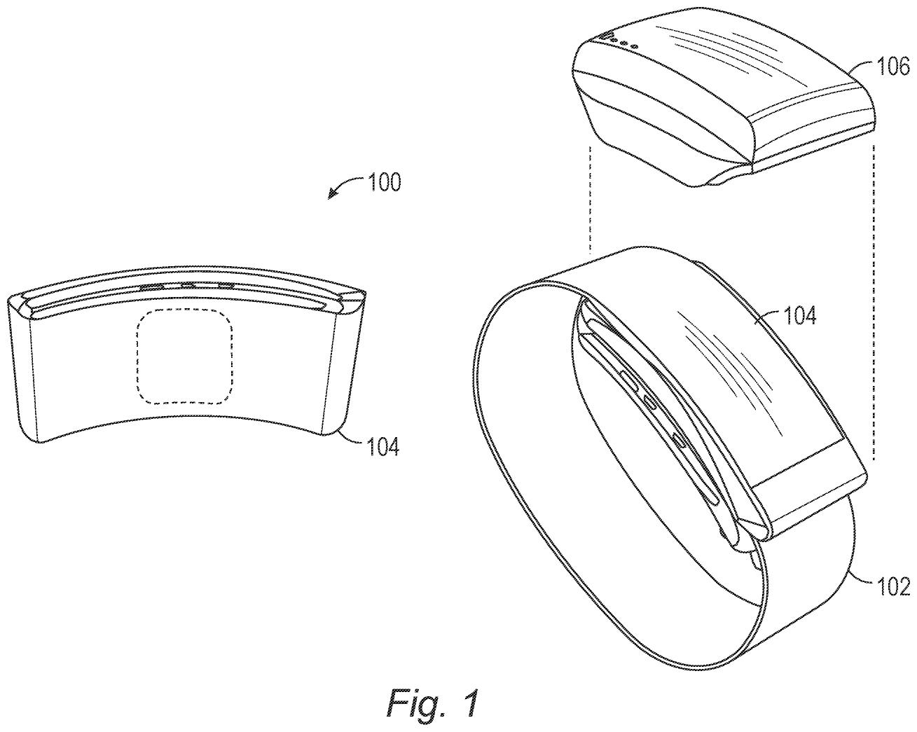

FIG. 1 shows a physiological monitoring device. The overall system 100 may include a device 104 (which may or may not include a display screen or other user interface) generally configured for physiological monitoring. The system 100 may further include a removable and replaceable battery 106 for recharging the device 104. A strap 102 may be provided, and may include any arrangement suitable for retaining the device 104 in a position on a wearer's body for acquisition of physiological data as described herein. For example, the strap 102 may include slim elastic band formed of any suitable elastic material, for example, a rubber, a woven polymer fiber such as a woven polyester, polypropylene, nylon, spandex, and so forth. The strap 102 may be adjustable to accommodate different wrist sizes, and may include any latches, hasps, clasps, and/or the like to secure the device 104 in an intended position for monitoring a physiological signal. While a wrist-worn device is depicted, it will be understood that the device 104 may be configured for positioning in any suitable location on a user's body, based on the sensing modality and the nature of the signal to be acquired. For example, the device 104 may be configured for use on a wrist, an ankle, a bicep, a chest, or any other suitable location(s), and the strap 102 may be, or may include a waistband or other elastic band or the like within an article of clothing or accessory.

The system 100 may include any hardware components, subsystems, and the like to provide various functions such as data collection, processing, display, and communications with external resources. For example, the system 100 may include a heart rate monitor using, e.g., photoplethysmography, electrocardiography, or any other technique(s). The system 100 may be configured such that, when placed for use about a wrist, the system 100 initiates acquisition of physiological data from the wearer. In some embodiments, the pulse or heart rate may be taken using an optical sensor coupled with one or more light emitting diodes (LEDs), all directly in contact with the user's wrist. The LEDs may be positioned to direct illumination toward the user's skin, and may be accompanied by one or more photodiodes or other photodetectors suitable for measuring illumination from the LEDs that is reflected and/or transmitted by the wearer's skin.

The system 100 may be configured to record other physiological parameters including, but not limited to, skin temperature (e.g., using a thermometer), galvanic skin response (e.g., using a galvanic skin response sensor), motion (e.g., using one or more multi-axes accelerometers and/or a gyroscope), and the like, as well environmental or contextual parameters such as ambient light, ambient temperature, humidity, time of day, and the like. The system 100 may also include other sensors such as accelerometers and/or gyroscopes for motion detection, and sensors for environmental temperature sensing, electrodermal activity (EDA) sensing, galvanic skin response (GSR) sensing, and the like.

The system 100 may include one or more sources of battery life, such as a first battery environmentally sealed within the device 104 and a battery 106 that is removable and replaceable to recharge the battery in the device 104. The system 100 may perform numerous functions related to continuous monitoring, such as automatically detecting when the user is asleep, awake, exercising, and so forth, and such detections may be performed locally at the device 104 and/or at a remote service coupled in a communicating relationship with the device 104 and receiving data therefrom. In general, the system 100 may support continuous, independent monitoring of a physiological signal such as a heart rate, and acquired data may be stored on the device 104 until it can be uploaded to a remote processing resource for more computationally expensive analysis.

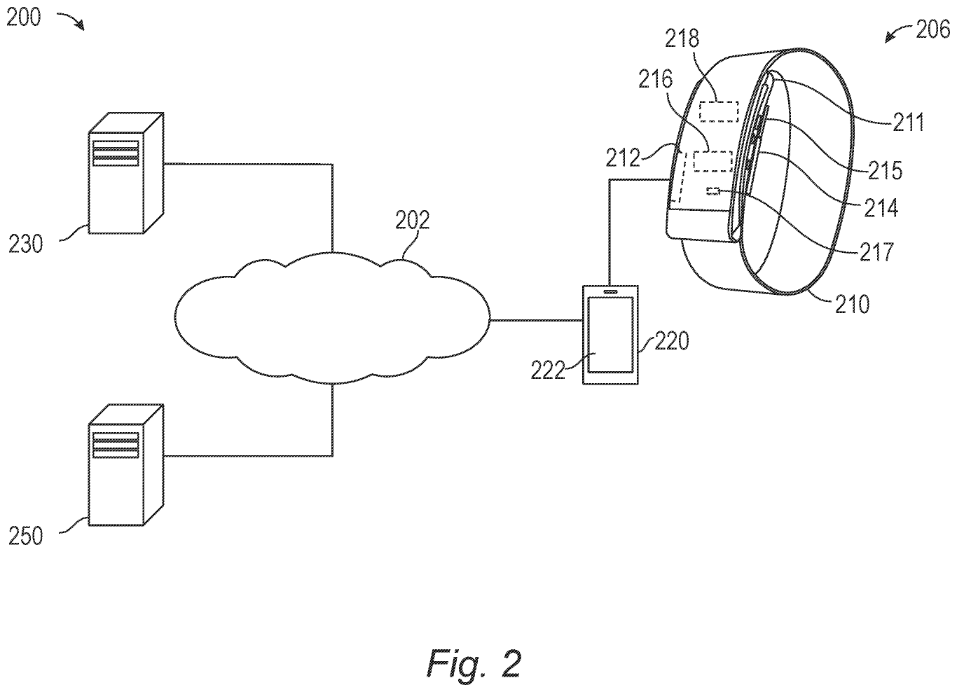

FIG. 2 illustrates a physiological monitoring system. More specifically, FIG. 2 illustrates a physiological monitoring system 200 that may be used with any of the methods or devices described herein. In general, the system 200 may include a physiological monitor 206, a user device 220, a remote server 230 with a remote data processing resource (such as any of the processors or processing resources described herein), and one or more other resources 250, all of which may be interconnected through a data network 202.

The data network 202 may be any of the data networks described herein. For example, the data network 202 may be any network(s) or internetwork(s) suitable for communicating data and information among participants in the system 200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 200. This may also include local or short range communications networks suitable, e.g., for coupling the physiological monitor 206 to the user device 220, or otherwise communicating with local resources.

The physiological monitor 206 may, in general, be any physiological monitoring device, such as any of the wearable monitors or other monitoring devices described herein. Thus, the physiological monitor 206 may generally be shaped and sized to be worn on a wrist or other body location and retained in a desired orientation relative to the appendage with a strap 210 or other attachment mechanism. The physiological monitor 206 may include a wearable housing 211, a network interface 212, one or more sensors 214, one or more light sources 215, a processor 216, a haptic device 217 (and/or any other type of component suitable for providing haptic or other sensory alerts to a user), a memory 218, and a wearable strap 210 for retaining the physiological monitor 206 in a desired location on a user.

In general, the physiological monitor 206 may include a wearable physiological monitor configured to acquire heart rate data and/or other physiological data from a wearer. More specifically, the wearable housing 211 of the physiological monitor 206 may be configured such that a user can acquire heart rate data and/or other physiological data from the user in a substantially continuous manner. The wearable housing 211 may be configured for cooperation with a strap 210 or the like, e.g., for engagement with an appendage of a user.

The network interface 212 may be configured to coupled one or more participants of the system 200 in a communicating relationship, e.g., with the remote server 230, either directly, e.g., through a cellular data connection or the like, or indirectly through a short range wireless communications channel coupling the physiological monitor 206 locally to a wireless access point, router, computer, laptop, tablet, cellular phone, or other device that can relay data from the physiological monitor 206 to the remote server 230 as necessary or helpful for acquiring and processing data.

The one or more sensors 214 may include any of the sensors described herein, or any other sensors suitable for physiological monitoring. By way of example and not limitation, the one or more sensors 214 may include one or more of a light source, an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, a temporal sensor, an electrodermal activity sensor, and the like. The one or more sensors 214 may be disposed in the wearable housing 211, or otherwise positioned and configured for capture of data for physiological monitoring of a user. In one aspect, the one or more sensors 214 include a light detector configured to provide data to the processor 216 for calculating a heart rate variability. The one or more sensors 214 may also or instead include an accelerometer configured to provide data to the processor 216, e.g., for detecting activities such as a sleep state, a resting state, a waking event, exercise, and/or other user activity. In an implementation, the one or more sensors 214 measure a galvanic skin response of the user.

The processor 216 and memory 218 may be any of the processors and memories described herein, and may be suitable for deployment in a physiological monitoring device. In one aspect, the memory 218 may store physiological data obtained by monitoring a user with the one or more sensors 214. The processor 216 may be configured to obtain heart rate data from the user based on the data from the sensors 214. The processor 216 may be further configured to assist in a determination of a condition of the user, such as whether the user has an infection or other condition of interest as described herein.

The one or more light sources 215 may be coupled to the wearable housing 211 and controlled by the processor 216. At least one of the light sources 215 may be directed toward the skin of a user's appendage. Light from the light source 215 may be detected by the one or more sensors 214.

The system 200 may further include a remote data processing resource executing on a remote server 230. The remote data processing resource may be any of the processors described herein, and may be configured to receive data transmitted from the memory 218 of the physiological monitor 206, and to process the data to detect or infer physiological signals of interest such as heart rate, heart rate variability, respiratory rate, pulse oxygen, blood pressure, and so forth. The remote server 230 may also or instead evaluate a condition of the user such as a recovery state, sleep quality, daily activity strain, and any health conditions that might be detected based on such data.

The system 200 may also include one or more user devices 220, which may work together with the physiological monitor 206, e.g., to provide a display for user data and analysis, and/or to provide a communications bridge from the network interface 212 of the physiological monitor 206 to the data network 202 and the remote server 230. For example, physiological monitor 206 may communicate locally with a user device 220, such as a smartphone of a user, via short-range communications, e.g., Bluetooth, or the like, e.g., for the exchange of data between the physiological monitor 206 and the user device 220, and the user device 220 may communicate with the remote server 230 via the data network 202. Computationally intensive processing, such as infection monitoring, may be performed at the remote server 230, which may have greater memory capabilities and processing power than the physiological monitor 206 that acquires the data.

The user device 220 may include any computing device as described herein, including without limitation a smartphone, a desktop computer, a laptop computer, a network computer, a tablet, a mobile device, a portable digital assistant, a cellular phone, a portable media or entertainment device, and so on. The user device 220 may provide a user interface 222 for access to data and analysis by a user, and/or to control operation of the physiological monitor 206. The user interface 222 may be maintained by a locally-executing application on the user device 220, or the user interface 222 may be remotely served and presented on the user device 220, e.g., from the remote server 230 or the one or more other resources 250.

In general, the remote server 230 may include data storage, a network interface, and/or other processing circuitry. The remote server 230 may process data from the physiological monitor 206 and perform infection monitoring/analyses or any of the other analyses described herein, and may host a user interface for remote access to this data, e.g., from the user device 220. The remote server 230 may include a web server or other programmatic front end that facilitates web-based access by the user devices 220 or the physiological monitor 206 to the capabilities of the remote server 230 or other components of the system 200.

The other resources 250 may include any resources that can be usefully employed in the devices, systems, and methods as described herein. For example, these other resources 250 may include without limitation other data networks, human actors (e.g., programmers, researchers, annotators, editors, analysts, and so forth), sensors (e.g., audio or visual sensors), data mining tools, computational tools, data monitoring tools, algorithms, and so forth. The other resources 250 may also or instead include any other software or hardware resources that may be usefully employed in the networked applications as contemplated herein. For example, the other resources 250 may include payment processing servers or platforms used to authorize payment for access, content, or option/feature purchases, or otherwise. In another aspect, the other resources 250 may include certificate servers or other security resources for third-party verification of identity, encryption or decryption of data, and so forth. In another aspect, the other resources 250 may include a desktop computer or the like co-located (e.g., on the same local area network with, or directly coupled to through a serial or USB cable) with a user device 220, wearable strap 210, or remote server 230. In this case, the other resources 250 may provide supplemental functions for components of the system 200.

The other resources 250 may also or instead include one or more web servers that provide web-based access to and from any of the other participants in the system 200. While depicted as a separate network entity, it will be readily appreciated that the other resources 250 (e.g., a web server) may also or instead be logically and/or physically associated with one of the other devices described herein, and may for example, include or provide a user interface 222 for web access to a remote server 230 or a database in a manner that permits user interaction through the data network 202, e.g., from the physiological monitor 206 or the user device 220, with processing and data resources of the remote server 230.

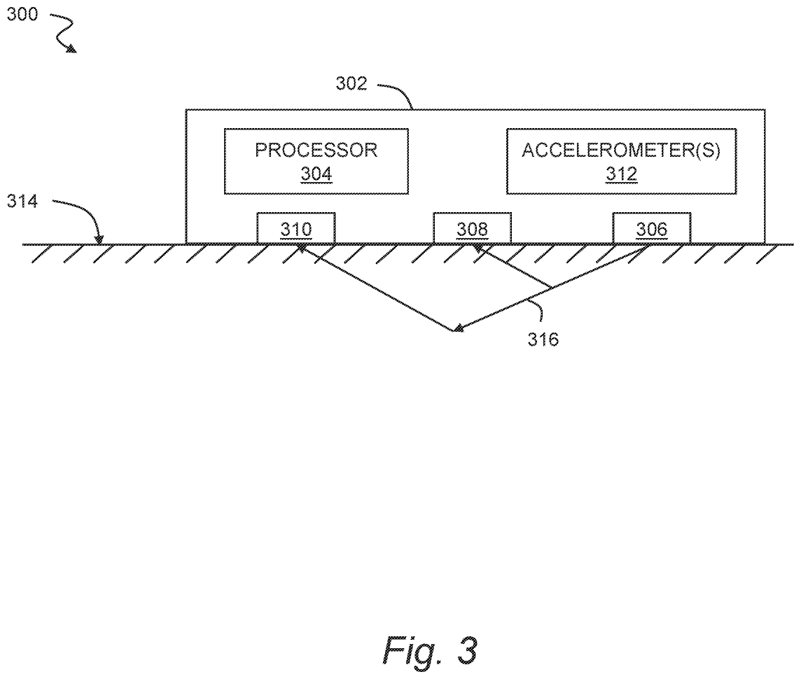

FIG. 3 shows a sensing system. In general, the system 300 may include a physiological monitor 302 including a processor 304, a light source 306, a first photodetector 308, a second photodetector 310, an accelerometer 312, and any other hardware or other components and systems suitable for physiological monitoring as described herein. The physiological monitor 302 may be positioned for use against a skin 314 of a user (or more specifically, against and/or adjacent to a surface of the skin 314) where the light source 306 and sensors 308, 310 can contact the skin 314 (or otherwise be in desired proximity to the skin 314) for acquisition of physiological data. It will be understood that, unless otherwise noted, when the physiological monitor 302 (or a component thereof) is described herein as “against” or “on” or in “contact” with the skin 314 of a user (or similarly described), this shall include implementations where the physiological monitor 302 (or a component thereof) is physically touching the skin 314 of the user, and implementations where the physiological monitor 302 (or a component thereof) may not be physically touching the skin 314 of the user but rather it is in close enough proximity to the skin 314 of the user for physiological monitoring as described herein. For example, in some implementations, a housing of the physiological monitor 302 touches the skin 314 of a user, but sensing components (e.g., the light source 306 and sensors 308, 310) are disposed at a relatively small separation from the skin 314 of the user that is sufficient for physiological monitoring as described herein. Further, although not depicted, it will be understood that the physiological monitor 302 will generally be retained in a position using any of the straps, garments, or the like described herein.

The processor 304 may be any microprocessor, microcontroller, application specific integrated circuit, or other processing circuitry suitable for controlling operation of the physiological monitor and acquiring physiological data.

The light source 306 may include one or more light emitting diodes or other sources of illumination, and may be positioned within the physiological monitor 302 such that, when placed for use on the skin 314, the light source 306 directs illumination toward the skin 314 as indicated by an arrow 316. Where the light source 306 provides non-uniform directional lighting, the peak intensity may be directed normal to an intended contact surface, or angled toward the sensors 308, 310, or in some other orientation consistent with acquisition of reflected/transmitted light at the sensors 308, 310. In one aspect, the light sensors may include light emitting diodes that emit light in the infrared or near infrared wavelength ranges, which can provide desirable light transmission through human skin, facilitating low-power transmission of measurable illumination to the sensors 308, 310.

The sensors 308, 310 may be positioned to contact the skin 314 when the physiological monitor 302 is placed for use on this skin 314, so that the sensors 308, 310 can capture illumination reflected and/or transmitted by the skin 314 from the light source 306. In general, the sensors 308, 310 may include photodiodes, photodetectors, or any other sensor(s) responsive to illumination from the light source 306. This may include broadband optical sensors, narrowband optical sensors, filtered sensors, or the like. In general, the first sensor 308 may be positioned closer to the light source 306 than the second sensor 310 to facilitate detection of differential intensity in the measured wavelength(s). For example, the first sensor 308 may be positioned about 1-4 millimeters from the light source 306 and the second sensor 310 may be positioned about 2-8 millimeters from the light source, or about twice as far as the first sensor 310 from the light source 306. Other configurations and spacings are also or instead possible.

For example, other spacings may also or instead be used depending on, e.g., the intensity of the light source 306, the sensitivity of the sensors 308, 310, the contact force of the physiological monitor 302 on the skin 314, the degree of incursion of ambient light, and so forth. In one aspect, the sensors 308, 310 may be linearly arranged in a straight line away from the light source 306. While this provides consistency in comparative measurements, it is not strictly required, and the sensors 308 may be displaced in any directions away from the light source 306 provided they both contact the skin 314 in a manner that permits capture of light through the skin 314 from the light source 306. In another aspect, the physiological monitor 302 may include one or more other light sources and/or light sensors, which may be arranged to improve accuracy and/or provide redundancy for the contact detection. This may include light sources/sensor using different ranges of wavelengths, different patterns of illumination, and so forth. Additional sensors (e.g., similar to the two sensors 308, 310) may be added at different, greater distances from the light source 306. Such additional sensors may permit the capture of light traveling through the skin 314 at different depths and provide additional differential information. In another aspect, the two sensors 308, 310 may be positioned at different distances from a perimeter of the physiological monitor 302 so that the sensors 308, 310 can acquire differential intensity values for ambient light incident on the skin and transmitted through the skin to the sensors 308, 310.

In operation, the processor 304 may acquire raw intensity data from the sensors 308, 310, and/or the processor 304 may calculate relevant features useful for determining whether the physiological monitor 302 is placed for use on the skin 314. For example, an averaging function may be applied to measured intensity to provide, e.g., a five second intensity average from each sensor 308, 310, in order to mitigate erroneous detections based on transient lighting patterns as a device is moved, removed, temporarily displaced from a wrist (or other portion of a wearer's body), and so forth. The processor 304 may then evaluate whether the physiological monitor 302 is positioned for use on the skin based on, e.g., a ratio or other function of intensity at the first sensor 308 to intensity at the second sensor 310.

The accelerometer 312 may include, e.g., one or more single axis or multi-axis accelerometers, which may usefully measure motion of the physiological monitor 302 to provide an input to a transition detection. For example, motion of the physiological monitor 302 may be used to trigger testing of whether the physiological monitor 302 has been placed on the wrist (or other portion of a wearer's body). This can advantageously provide a low-power, threshold test before driving the light source 306 and measuring light intensity with the sensors 308, 310.

More generally, the physiological monitor 302 may include any additional components, subsystems, and the like suitable for detecting whether the physiological monitor 302 is placed for use as described herein, and for supporting various modes of physiological monitoring and contextual data acquisition.

According to the foregoing, there is described herein a device including a physiological monitor having a light source providing a source of illumination, a first sensor responsive to the illumination and positioned a first distance from the light source, and a second sensor responsive to the illumination from the light source and positioned at a second distance greater than the first distance from the light source. The device may also include a processor within the physiological monitor, the processor configured to activate the light source to provide the illumination, to measure a first intensity of the illumination at the first sensor, to measure a second intensity of the illumination at the second sensor, and to determine whether the physiological monitor is positioned for use on a skin of a user based on a comparison of the first intensity to the second intensity.

The device may also or instead include an accelerometer 312, where the processor 304 is configured to activate the light source 306 in response to motion detected by the accelerometer 312. The comparison may be a function of the first intensity and the second intensity, and may be based, for example, on a ratio of the first intensity to the second intensity, a range between the first intensity and the second intensity, and so forth. In this context, it will be understood that the ratio need not be an isolated ratio of the two intensities, and may also or instead include any function where an increase in the intensity measured at the second sensor 310 relative to the first sensor 308 is correlated to better contact with the skin 314. In one aspect, the comparison may be performed by a state machine. The state machine may include at least one state transition based on movement detected with an accelerometer 312 of the physiological monitor 302, e.g., where the detected motion serves as a trigger to transition from an idle or off-body state to a testing state for contact. In another aspect, the state machine may include at least one state transition based on a capacitive sensor of the physiological monitor 302, e.g., where optical contact sensing is used in combination with capacitive sensing.

In another aspect, the comparison may be performed by a machine learning algorithm trained with data labeled as on skin and off skin, or for a wrist-worn device, with data labeled as on-wrist or off-wrist, or the like. For example, the machine learning algorithm may be trained to distinguish between on-body and off-body states using sensor data acquired when the physiological monitor 302 is off-body (e.g., labeled “off”) and other sensor data acquired when the physiological monitor 302 is on-body (e.g., labeled “on”). The sensor data for this training data set may also or instead include data from other sensors (e.g., a capacitive sensor, an accelerometer, ambient light data, etc.), as well as derived metrics such as an interval average for the intensity, a ratio of intensities, a range of intensities, differences between intervals, and so forth. By way of example, changes in ambient light detection may be used as a factor in training data.

The physiological monitor 302 may include any physiological monitor. In one aspect, the techniques described herein may be advantageously incorporated into a monitor that already provides an optical source and sensing hardware, such as a heart rate monitor using photoplethysmography or a pulse oximetry monitor for measuring peripheral oxygen saturation.

As described herein, the processor 304 may be configured to, in response to determining that the physiological monitor 302 is positioned for use on the skin 314 of the user, perform a physiological monitoring process with the physiological monitor 302. That is, the processor 304 may initiate monitoring in response to a detection that the monitor is placed for use on the user's body. The processor 304 may also or instead be configured to, in response to detecting a transition from a state of positioned for use to not positioned for use, stop a physiological monitoring process by the physiological monitor 302.

At least one of the first intensity and the second intensity may be measured in an infrared range. The light source 306 may include an infrared light emitting diode. The first intensity may be measured as an average over an interval, such as an interval of at least three seconds or an interval of about five seconds, or some other length. The second intensity may also or instead be measured as an average over an interval. The interval may be at least three seconds or about five seconds, or some other length.

The physiological monitor 302 may also or instead more generally characterize the nature of the material against which it is placed. That is, skin 314 is composed of multiple layers of different materials, and differentials from the sensors 308, 310 may provide correlations to known material properties of layers of the skin including the epidermis, dermis, and the hypodermis. Additional sensors (e.g., similar to the sensors 308, 310) positioned at greater distances from the source 306 may provide additional differential measurements and correlations, which can allow the resolution of properties of additional layers of skin 314 at different depths.

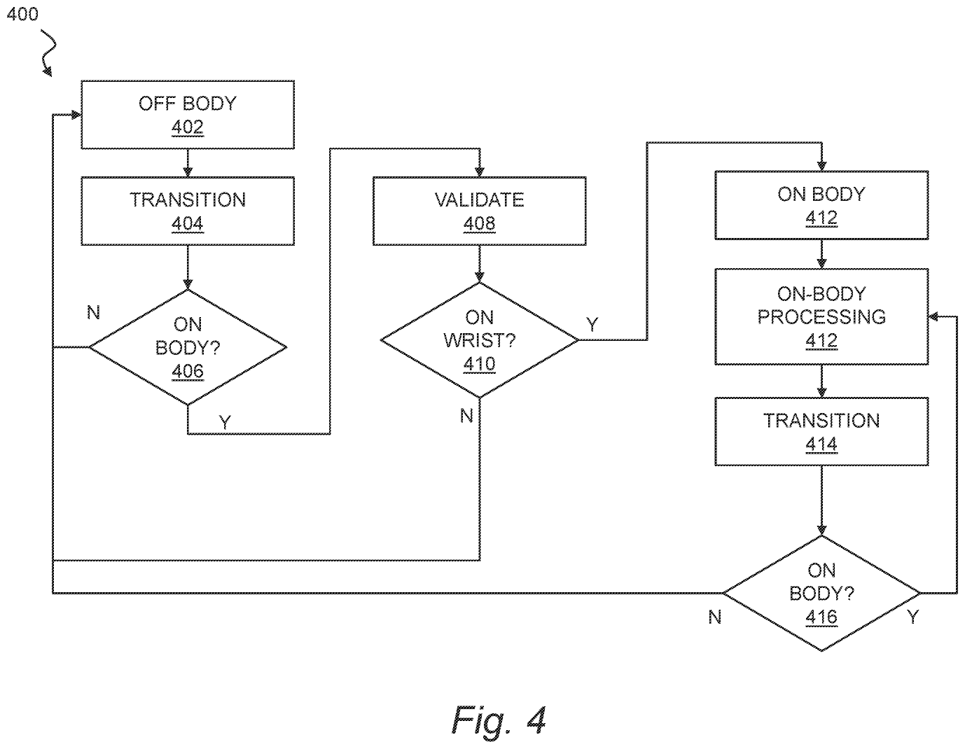

FIG. 4 shows a flow chart of a method for detecting device position. In general, the techniques described herein may use a ratio of light intensity at two sensors, such as those described above, to evaluate whether a physiological monitor or the like is properly positioned for use on a user's body. This optically-based detection technique may be deployed within a more general on/off detection algorithm to accurately determine when a device is on the body— e.g., when the physiological monitoring device should be actively monitoring physiological data—and when the device is off the body, e.g., when physiological monitoring should be idle. As a significant advantage, properly identifying whether the device is on the body and placed for use permits conservation of power and processing resources for periods during which accurate physiological data can be acquired, and improves data integrity by avoiding data acquisition during periods where meaningful physiological data is not available. It will be understood that, where the device is designed for use in a specific body location, the algorithm may instead determine whether or not the device is positioned at that location. For example, the method 400 may be specifically adapted to determine whether a device is on-wrist or off-wrist, on-ankle or off-ankle, or otherwise properly positioned for use on the body.

As shown in step 402, the method 400 may include operating a device, such as any of the physiological monitoring devices described herein, in an “off body” mode. This may occur when the device is initially powered on, or when the device has transitioned to an off body mode due to prior operating conditions. In this mode, the device will generally not be actively monitoring for physiological data. That is, the device will not power its sensing systems, acquire raw data, and/or process the raw data to derive physiological measurements. The device may instead perform other functions, such as data communications, firmware updates, and monitoring for state transitions as described herein. However, the device will not actively acquire physiological data in any continuous and/or intermittent manner.

As shown in step 404, the method 400 may include detecting a transition event. This may be any event based on sensor data that indicates a possible transition to a different device location or use. For example, the device may monitor ambient light intensity, infrared light intensity (either from ambient sources, or from an adjacent, active illumination source), or the like with an optical sensor for a change in light intensity (e.g., a decrease in intensity due to occlusion of ambient light) that might be indicative of placement of the device for use. This may also or instead include monitoring for a sudden and/or substantial change in light intensity consistent with movement or repositioning of the device. The device may also or instead monitor for motion, e.g., from accelerometer data, gyroscope data, or signals from other motion sensors, in order to detect repositioning and/or reorientation of the device. For example, if the device is moved, placed on a surface, flipped over, or otherwise handled, there will typically be one or more spikes in sensed data from motion detectors such as accelerometers, gyroscopes, and so forth. The transition event may be any such spike in data, or combination of spikes in data, or other data discontinuity or the like indicative of a sensed transition in use or positioning of the device. Cyclic or periodic motions, such as those typical of exercise or extended activities such as walking, may also serve as a transition event, particularly when the device is in an off-body mode and the device appears to be on the body of a user who is exercising. At the same time, certain periodic motions are not necessarily indicative of an on-body device. For example, where a wearable device is in a pocket, or in a handbag that is being carried by a user, there may be periodic motion artifacts that indicate activity, but do not suggest that the wearable device is in a suitable position for acquiring physiological data. Any of these patterns may be used to filter motion data or other sensor data to determine when a transition event has occurred (or not occurred) for which an on-body detection is appropriate.

In one aspect, the transition event may be motion that is more specifically indicative of placement of the device for use. While the nature and scope of such movements may vary according to the desired degree of sensitivity, as well as a desired avoidance of false positives, suitable transition events will generally include transient movements (e.g., less than ten or twenty seconds) consistent with activities such as picking up the device and placing it on a limb or other body location. Similarly, this will generally exclude periodic, repeated movements consistent with, e.g., exercise, walking, eating, and so forth.

As shown in step 406, the method 400 may include, in response to detecting a transition event, evaluating whether the device is positioned on a user's body. For example, this may begin with activating a light source (also referred to herein as an illumination source), or continuing operation of the light source if it is already activated, and initiating an on/off detection based on analysis of measured optical signals. The on/off detection may employ any of the techniques described herein. In general, the detection technique may advantageously employ a light intensity measurement at two different sensors positioned on the device at different distances from the light source in order to test not just whether the device is in contact with a surface, but whether the device is in contact with a surface that appears to be human tissue.

The acquired optical signals may be processed using a variety of techniques. For example, in one aspect, the intensity measured at the sensors may be preprocessed, e.g., by averaging the intensities over intervals such as three seconds, four seconds, five seconds, or some other value between or outside these durations. These values, in raw or pre-processed form (or some combination of these) may then be applied to any suitable algorithm to evaluate whether the device is positioned for use. For example, the device may use a state machine or machine learning algorithm to process the data from the two sensors and determine whether the device appears to be positioned for use. In another aspect, the processor may apply a formula to the two sensor values such as a ratio of the values, a difference between the values, a relative difference between the values (e.g., by taking the difference between the values and dividing this quantity by one of the values), or some other formula useful for comparing data from the two sensors. The resulting quantity, referred to generally herein as a differential, may be compared to a threshold to determine whether the device is on-body or off body.

The method 400 may include continuously checking for an on-body condition for some predetermined period of time, or until the occurrence of some event, or some combination of these. For example, the method 400 may include waiting for an end to the spike in sensor data, and then waiting an additional interval during which sensor data is evaluated for device positioning. If, during this time, it is determined that the device is positioned for use based on the sensor data, then the method 400 may proceed to step 408 where the position is validated. If, after the conclusion of this time, it is determined that the device is not positioned for use based on the sensor data, then the method 400 may return to step 402, where the device remains in an idle state and does not monitor for physiological activity.

In one practical embodiment, an on/off-body algorithm may use the following streams of information to detect a transition event (and subsequently, validate position): a low-level background infrared (“IR”) channel (with near and far photodetectors so that a ratio or other differential can be calculated); an ambient light channel; and accelerometer or gyroscope data relating to movement. The background IR channel may be a channel that is configured differently than those used for heart rate or oxygen saturation. For heart rate and oxygenation, the system may use pairs of photodetectors (PDs) that have the same distance from corresponding light sources. For example, this may include one or more nearby photodiodes (“near PDs”), which may be equally distanced from a light source, and used in a PPG device for acquiring heart rate data. For oxygen saturation, this may include a set of more distant photodiodes (“far PDs”) that are each about the same distance from a light source, but a different (and farther) distance than the near PDs used for heart rate detection. For on/off body detection, a background IR channel with one near PD and one far PD may advantageously be used. The LED current in this channel may be relatively low, about 2-3 mA, as compared to significantly more current (e.g., 50 mA) for physiological monitoring channels. This advantageously permits the background IR channel used for on/off detection to be run in the background at a relatively small power penalty.

From these data streams, the following metrics may be generated and used in the algorithm: IR near PD average (about 5 seconds); IR far PD average (about 5 seconds); ratio of IR near PD average to far PD average (about 5 seconds); range (max-min) of IR near PD (about 1 second); range of accelerometer magnitude (from 3 axes X/Y/Z); and ambient light average (about 2 to about 10 seconds). Each of these metrics can take on a wide range of values when a monitor is off the body, but will tend to assume a narrower and more stable range of values when the device is on the body, e.g., placed for use in physiological monitoring. The bounds for these ranges may be empirically evaluated, and have been demonstrated to provide reliable on/off indications over a wide range of users and use cases. When most/all features fall in the range for being on-body, a reliable determination can be made, based on a single measurement, that the wearable device is on the user's body. However, a more stable and generally reliable determination may usefully be made based on a collection of measurements over time.

In general, there may be large spikes in the optical IR mean at various intensity values when off-body, e.g., as a result of widely varying incident light on the exposed optical detectors. However, when the device is on the body, a distribution of individual intensity measurements may appear roughly gaussian. From this characteristic behavior, a threshold for on-wrist detection may be based on intensity for a near PD. However, looking at a single feature such as this in isolation may be prone to false positives. Thus, multiple quantitative metrics may advantageously be evaluated in parallel, and added, differenced, ratioed, or otherwise compared to support a reliable conclusion concerning positional status of the wearable device.

For example, one useful metric is the ratio between the near PD average and the far PD average. As a significant advantage, this metric usefully distinguishes among a sensor array that is facing up (or otherwise exposed), a sensor array that is facing down on an opaque surface such as a table, and a sensory array that is properly placed for data acquisition on a user's body. In the case of an exposed sensor array, little, if any, illumination from a light source will reflect back to the detectors, and the ratio of near and far detection intensities should be close to one. Conversely, for an opaque or other non-transmissive material in loose contact with the sensor array, the drop off in intensity with distance should be very high, and the far-to-near ratio should approach zero. However, for a moderately transmissive material such as skin and underlying tissue, the ratio will have an intermediate, and generally consistent drop off in intensity associated with the source-detector distance. Since the distance between the two photodiodes (or other optical sensors) is fixed for a particular sensor array, a relative intensity of photodiode measurements can be predicted based on known diffusion/scattering/attenuation patterns for human tissue, and/or empirically confirmed for an individual or for a group of users. In practice, this ratio may be affected by many factors such as pressure, fit, skin variability, and so forth. However, an expected value may be established for an individual or for a population of users, and a range may be developed around the expected value suitable for accommodating different users, different strap tensions, and the like. Thus, for example, a lower threshold may be a limit between 0.3× and 0.5× of the expected value of a ratio of averaged optical measurements at two differently located detectors, and an upper threshold may be a limit between 2× and 3× of the expected value.

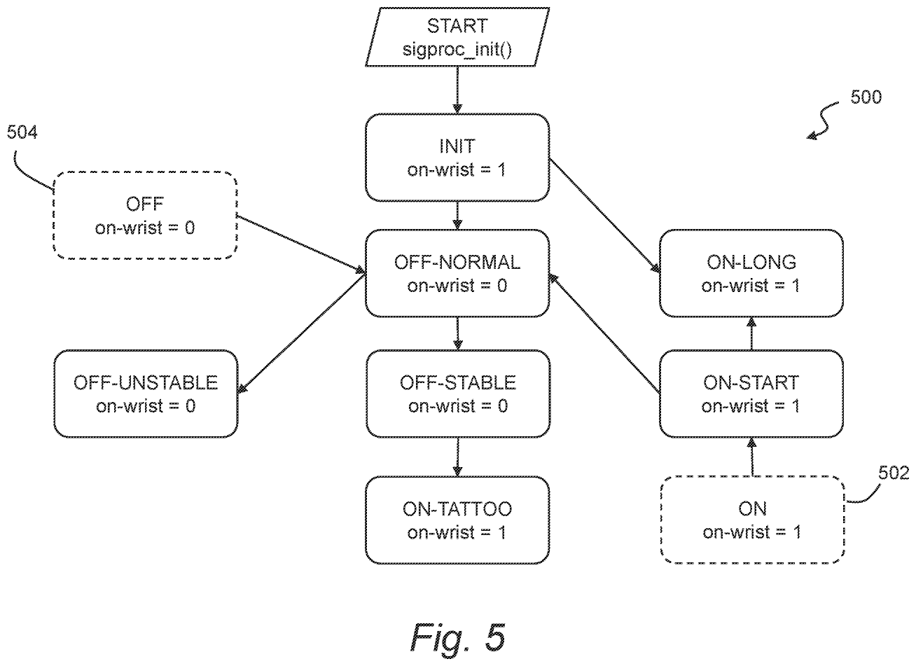

These and other metrics may provide a useful measure for instantaneous evaluation of the on/off state of a wearable device, as well as for validation as discussed below. However, the general detection of ‘on’ and ‘off’ states may be improved by deploying these measurements in the context of a method that accounts for different usage scenarios and detection patterns. A finite state machine (FSM) or similar programming construct may be used that operates on combinations of these and other metrics, and adapts a monitoring state based on current or historical usage information. The FSM may move between various states of “on-body”and “off body” monitoring by looking at the time of a current state, the stability of measured values, the presence and nature of motion, and so forth. An example of such a state machine, within the context of a more comprehensive on-off detection strategy, is described in reference to FIG. 5 below.

As shown in step 408, the method 400 may include, when it is determined that the device is positioned on the body for use, entering a validation state. In this state, a processor may continue to monitor sensor data to verify positioning of the device, e.g., by averaging a number of results over a predetermined time window, or otherwise checking for substantially consistent results over a number of measurements. In one aspect, the processor may initiate physiological monitoring during this validation state, in another aspect, the processor may wait until a validation period has been completed before initiating an acquisition of physiological data.

In one aspect, the validation of positioning for use may be based on the techniques described above, e.g., by providing illumination from the light source and evaluating a differential of the sensed signals. In another aspect, the validation may be based on other related data. For example, variations in the received infrared signal may be compared to device motion, and if the variations in signal strength seem unusually large compared to the displacement of the device, this may indicate that the device is stowed in a pocket or bag rather than positioned on the user's wrist. This validation state may last for any interval suitable for accurately assessing device positioning. For example, the validation state may last for 2-20 minutes, during which time the device may periodically test for positioning on the skin as described herein. This approach ensures that the device is not accidentally placed in a physiological monitoring mode, or on-body mode, inadvertently due to an unusual and transient sequence of triggering events.

As a significant advantage, this technique may also or instead be used to ensure that the wearable device is properly positioned and suitably engaged with the tissue of a user to perform accurate physiological monitoring. Although not illustrated in FIG. 4, it will be understood that suitable, corresponding notifications may be provided to the user. For example, if the device is placed around a wrist such that it is generating suitable motion artifacts, but the optical sensors are not obtaining signals indicative of proper placement or tissue engagement (based on the thresholds described above), the user may be notified to adjust positioning of the device for secure engagement. This notification may be provided as an audible, tactile, and/or visual output of the device, and/or as a message to a smartphone or other computing device associated with the user.

As shown in step 410, if during the validation state, the positioning of the device on the body is verified, then the method 400 may proceed to step 412 where the device enters an on-body state. After entering the on-body state, the method 400 may include any physiological monitoring for which the device is configured, and may include gathering and reporting physiological data such as heart rate, heart rate variability, body temperature, respiratory rate, oxygen saturation, and the like.

As shown in step 414, the method 400 may include additional processing associated with being on the body and positioned for use. For example, this may include alerting a user to the on-body detection by illuminating a light emitting diode on the wearable device, providing a notification on an associated computing device, vibrating the wearable device with a haptic device, or otherwise indicating proper positioning for use. In one aspect, this may include, in response to determining that the physiological monitor is positioned for use on a skin of a user, providing a notification to the user that the device is ready for use, such as a haptic vibration, an LED illumination (e.g., green, indicating readiness for use), an audio notification, and so forth. More generally, any audio, visual, programmatic, or other output may be provided based on the detection of proper positioning in the on-body state. Where audio-visual output is employed, the output device is preferably positioned where it can be noticed by a user. For example, a light emitting diode that is used to indicate proper on-body positioning is preferably positioned on a surface of the wearable device that is visible to the user, e.g., readily visible on an exposed surface, when the wearable device is placed for use. Additional processing may also or instead include the initiation or continuation of physical monitoring using any suitable combination of sensors and processing for acquiring contact-based physiological measurements or the like as described herein.

As shown in step 414, the method 400 may include detecting a second transition event, such as an event indicative of removal of the device from the body. The second transition event may, for example, include an extended period of inactivity, a loss of, or inconsistency in, measured physiological signals, a spike in optical signal indicative of ingress of ambient light, and so forth.

As shown in step 416, the method 400 may include, in response to detecting a second transition event indicative of possible removal of the device, performing an on/off detection test using any of the techniques described herein. It will be understood that, when responding to an event indicating a possible removal of a device (as distinguished from a possible placement of the device, as contemplated in steps 404-406), a more permissive filter may be applied. For example, the ratio of measured light intensities, as well as minima and maxima for detecting device positioning, may be quantitatively relaxed in this case, based on an assumption that the device has remained in a position for data acquisition unless there is substantial contrary quantitative data. If an on-body detection is made, the method 400 may return to step 412 where additional on-body processing (e.g., including continued physiological monitoring) may be performed. If an on-body detection is not made, then the method 400 may return to step 402, where the device will operate in an off-body state and resume testing for a transition to an on-body state.

Although not depicted herein, it will be understood that a variety of other sensed conditions or triggering events may be used to transition from the on-body state to the off body state (and vice versa). Similarly, other sensed conditions or triggering events may be used to alter flow control for the method 400, for example, by terminating validation when a sustained increase in detected ambient light indicates an off body position for the device, or by transitioning out of validation to on-body when detected motion indicates a known type of exercise or physical activity.

FIG. 5 shows a state machine implementing an on/off detection process. In general, an on/off detection algorithm may run continuously in the background. A general goal of this algorithm is to determine if the physiological monitoring device is on the body of a user and positioned for physiological monitoring, or off the body of the user. When the wearable device is off-body, the device status can be reported or flagged so that other processes or signal processing algorithms that are only needed during physiological monitoring can be bypassed. This includes, e.g., algorithms supporting collection, detection, and/or processing of, e.g., heart-rate data and oxygen saturation data, and so forth. When the wearable device is on the body, these algorithms may be run in order to acquire corresponding physiological data.

The state machine illustrated in FIG. 5 may be used in combination with the other techniques described herein to more generally monitor the on/off status of a wearable device. In general, the state machine 500 may be used to implement the methods described herein, and may generally manage various special use cases, interim states, and the like in order to ensure proper on/off detection over a wide range of possible circumstances. In FIG. 5, the on/off state is indicated by an “on-wrist” flag, which is equal to either 1, indicating that the wearable device is in position for use, or 0, indicating that the wearable device is not in position for use. The value of this flag will change as the state machine 500 transitions from state to state based on detected conditions for the wearable device. For completeness, it will also be noted that when the device is in a stable state for an extended period of time, the device may transition to a suitable long term state such as a long term on state 502 or a long term off state 504. While transitions to these states are not indicated (because they may be reached from numerous other states), the transitions from these long term states will consistently return to specific monitoring states in the state machine 500, all as illustrated in FIG. 5. It will be noted that the term “state” as used herein, may be used to refer to a programmatic or monitoring state of the wearable device, e.g., any of the monitoring states illustrated in the method 400 described above with reference to FIG. 4 or the state machine 500 with reference to FIG. 5. This may include a programmatic on-body or off-body state as indicated by a flag or other data structure on the wearable device. However, the term “state” may also refer to a physical state of the wearable device, e.g., as either on the body of a user or off the body of the user. An effort has been made to refer to the physical state explicitly where appropriate, however the term “state” may in general refer to either or both of these states unless a specific meaning is otherwise provided or clear from the context.

Out of a reset (e.g., a reboot), the system may have no information about the previous state. By default, the device may reboot assuming that the device is on-body, and in the initial state, the system may enter an initialization state (“INIT”) where the device assumes an on-body state until enough data (e.g., 10 seconds worth) is available to determine otherwise. If it appears that the device is off-body, the device may transition to an off-body state (“OFF-NORMAL”); if it appears that the device is on-body, the device may remain in the on-body state (“ON-LONG”).

There are a number of off-body scenarios of interest, for purposes of correctly evaluating state. For example, this may include a physical state where the device is face-up or to the side, a physical state where the device is face-down on a hard surface, a physical state where the device is face-down on a permeable surface (e.g., a blanket), or a physical state where the device is in a pocket or a bag and moving around. There are also a number of on-body scenarios of interest. For example, this may include a physical state where the user moving around (e.g., typing when the device is on-wrist), a physical state where the user is asleep, a physical state where the user has the strap on a tattoo, and so forth. The on/off detection algorithm may be adapted to properly respond across these various use cases. However, some are more difficult than others to distinguish from one another, and tradeoffs may thus be made. In general, there may be a preference to preserve an on-body state, particularly for a continuously wearable monitoring device, where the device is expected to be on the user a majority of the time, and also more particularly where false negatives may result in a poor user experience due to gaps in the physiological data. However, false positives can also be detrimental when they cause spikes in the measured heart rate (e.g., picking up a light signal, or the like) and create downstream errors in calculated metrics such as physical strain. The state machine 500 in FIG. 5 attempts to address these trade-offs in a number of usage scenarios.

Assuming the device is off the body and is being placed on the body, for example, a transition event may be detected by looking at ambient light, infrared light, and motion (e.g., indicating that the device was moved and put on a surface). Around this transition event, the device may check for a change in metrics (e.g., ratio of intensity at two different detectors positioned at different distances from a light source) to see if they indicate that the device is on-body. If so, the state machine 500 may transition to a starting state for being worn (“ON-START”). In this ON-START state, the light sources may turn on and the on-wrist flag may be set to 1 (indicating on-body). This physical on-body state may then be validated over some subsequent window of time, e.g., 2-20 minutes, where the device is periodically checked to ensure that the measured metric(s) are remaining within a valid or expected range. In one aspect, this may include ensuring that the device has not been placed on a non-body surface with similar optical characteristics, e.g., by checking for periodic motion by the device over the validation window, or checking that a ratio of near and far intensity measurements is within a narrow range of expected results. This may also include comparing a range of motion relative to variations in the measured optical signal. For example, if there is small movement accompanied by large changes in a measured infrared detection signal, this may indicate that the device is likely in a pocket or bag rather than on the user's body. Conversely, if the optical measurement metrics remain stable and movement is within expected ranges, it can be confidently concluded that the device is on a human body. After validation over some predetermined time period, the device may transition to the ON-LONG state, where there is a confidence that the device is on-body. In this case, the device can be assumed to remain on the body until there are specific quantitative indicators to the contrary. If the device becomes unstable or falls out of an on-body range, then the device may transition to the OFF-NORMAL state.

The transition event may be a characteristic waveform from some sensor or collection of sensors that arises when a device physically moves from an off-body position to an on-body position. For example, this movement will naturally produce a decrease in the ambient light (if there is ambient illumination) as an optical sensor moves from open space to a position against the user's skin. There will also be some characteristic movement that can be detected with one or more motion sensors of the device. When these or other characteristic changes are detected, the collective changes may be a transition event demarcating a physical state change for the device that can be identified with confidence.

In some cases, a transition event, such as a spurious signal or unexpected change in measured values or calculated metrics, may suggest a transition. However, the decision to transition to a different monitoring state may be deferred until additional quantitative evidence can be acquired. Thus, transitions between states in the state machine 500, or more generally, in the monitoring states of the device, may be deferred in many cases to permit additional data acquisition and assessment to improve the confidence in the detection of a change in the physical state of the device.

Similarly, there are other special conditions that might usefully be modeled by additional states in the state machine 500. For example, when the device is in a pocket or bag and moving around, calculated metrics may periodically fall in range and out of range for being on the body, however, it would be undesirable to frequently toggle between an on state and an off state in a correspondingly small window of time. Thus, after an initial off-body detection (e.g., OFF-NORMAL), the device may transition to one of two alternate states in the state machine 500, depending on measured conditions. When measured or calculated values for the device are unstable, e.g., varying significantly over a short period of time, or varying excessively relative to a magnitude of detected movement, the state machine 500 may transition from OFF-NORMAL to OFF-UNSTABLE, and enter a state that continues to monitor for possible transitions, but algorithmically favors remaining in an off-body state. The state machine 500 may periodically return to the OFF-NORMAL state, or some other state, e.g., after a predetermined period of time, or by another transition event such as substantial changes in detected ambient light or a characteristic movement (or some combination of these). In the event that there is not excessive variation in measured parameters, the state machine 500 may instead transition to an OFF-STABLE state where continued monitoring proceeds on an assumption that the device is off the body.

In the case of the OFF-STABLE state, where the device processes data based on an assumption that the device is off-body, there is one additional use case that can usefully be accommodated. For tattooed users, calculated optical metrics may not fall in the expected range. This can prevent the device, when positioned for use on such a user, from transitioning within the context of the state machine 500 to an on-body state. To accommodate this situation, a separate state transition may be provided from OFF-STABLE to ON-TATTOO. In this case, the transition to an on-body state may be slower, e.g., requiring additional data acquisition and/or an extended window of time in order to accumulate additional evidence supporting a conclusion that the device is on a body with tattoos. In particular, the device may transition from OFF-STABLE to ON-TATTOO when, e.g., over some predetermined window of time, one or more calculated metrics are out of range, but there is (a) some motion detected, and (b) the infrared intensity range remains stable. The ON-TATTOO state may also include a final validation step based on an attempt to acquire a valid heart rate signal over some predetermined interval. Once in this ON-TATTOO state, the state machine will remain in an on-body state until a suitable transition event (e.g., increase in ambient light) indicates removal.

More generally, any arrangement of monitoring states and transitions consistent with accurately detecting on and off states for a wearable device may be deployed in a state machine or similar programming structure or the like to facilitate detection of the physical state of the wearable device, and more particularly whether the wearable device is on the body or off the body.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software.

Thus, in one aspect, each method described above, and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. The code may be stored in a non-transitory fashion in a computer memory, which may be a memory from which the program executes (such as random access memory associated with a processor), or a storage device such as a disk drive, flash memory or any other optical, electromagnetic, magnetic, infrared, or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

1. (canceled)

2. A computer program product comprising computer executable code embodied in a non-transitory computer readable medium that, when executing on one or more processors of a wearable device, causes the wearable device to perform the steps of:
detecting, with a motion sensor of the wearable device, a transition event indicative of placing the monitor for use on a user;
in response to detecting the transition event, performing the steps of:
providing illumination from a light emitting diode of the wearable device,
measuring a first intensity of the illumination with a first optical sensor a first distance from the light emitting diode,
measuring a second intensity of the illumination with a second optical sensor a second distance from the light emitting diode, wherein the second distance from the light emitting diode is greater than the first distance from the light emitting diode, and
determining whether the wearable device is positioned for use on a skin of the user based on a comparison of the first intensity to the second intensity; and
displaying an indicator to the user of whether the wearable device is positioned for use with a light emitting diode on the wearable device.

3. The computer program product of claim 2, wherein displaying the indicator includes illuminating the light emitting diode when the wearable device is properly positioned for use.

4. A method, comprising:
detecting a transition event indicative of placing a wearable monitor for use on a user;
in response to detecting the transition event, performing the steps of:
providing illumination from a light emitting diode of the wearable monitor,
measuring a first intensity of the illumination with a first optical sensor a first distance from the light emitting diode,
measuring a second intensity of the illumination with a second optical sensor a second distance from the light emitting diode, wherein the second distance from the light emitting diode is greater than the first distance from the light emitting diode, and
determining whether the wearable monitor is positioned for use on a skin of the user for acquisition of physiological data based on a comparison of the first intensity to the second intensity; and
displaying an indicator to the user of whether the wearable monitor is positioned for use.

5. A device, comprising:
a physiological monitor comprising a light source providing a source of illumination, a first sensor responsive to the illumination and positioned a first distance from the light source, and a second sensor responsive to the illumination from the light source and positioned at a second distance greater than the first distance from the light source; and
a processor within the physiological monitor, the processor configured to activate the light source to provide the illumination, to measure a first intensity of the illumination at the first sensor, to measure a second intensity of the illumination at the second sensor, and to determine whether the physiological monitor is positioned for use on a skin of a user based on a comparison of the first intensity to the second intensity.

6. The device of claim 5, further comprising an accelerometer, wherein the processor is configured to activate the light source in response to motion detected by the accelerometer.

7. The device of claim 5, further comprising a capacitive touch sensor, wherein the processor is configured to activate the light source in response to motion detected by the capacitive touch sensor.

8. The device of claim 5, wherein the processor is configured to activate the light source and measure illumination in response to detecting a motion of the physiological monitor indicative of placement for use.

9. The device of claim 5, wherein the comparison is one or more of: a function of the first intensity and the second intensity; based on a ratio of the first intensity to the second intensity; or based on a range between the first intensity and the second intensity.

10-11. (canceled)

12. The device of claim 5, wherein the comparison is performed by a state machine, the state machine including at least one state transition based on: movement detected with an accelerometer of the physiological monitor; or a capacitive sensor of the physiological monitor.

13. (canceled)

14. The device of claim 5, wherein the physiological monitor includes at least one of a heart rate monitor and a photoplethysmography system.

15. (canceled)

16. The device of claim 5, wherein the processor is configured to, in response to determining that the physiological monitor is positioned for use on the skin of the user, perform a physiological monitoring process with the physiological monitor.

17. The device of claim 5, wherein the physiological monitoring process includes one or more of heart rate monitoring and peripheral oxygen saturation monitoring.

18. The device of claim 5, wherein the processor is configured to, in response to detecting a transition from positioned for use to not positioned for use, stop a physiological monitoring process by the physiological monitor.

19. The device of claim 5, wherein at least one of the first intensity and the second intensity is measured in an infrared range.

20. The device of claim 5, wherein the light source includes an infrared light emitting diode.

21. The device of claim 5, wherein one or more of the first intensity and the second intensity is measured as an average over an interval.

22. The device of claim 21, wherein the interval is at least three seconds.

23. The device of claim 21, wherein the interval is about five seconds.

24-26. (canceled)

27. The device of claim 5, wherein the comparison is performed by a machine learning algorithm trained with training data that is labeled as on wrist or off wrist.

28. The device of claim 5, wherein the comparison is performed by a machine learning algorithm trained with data labeled as on skin or off skin.

29. The device of claim 28, wherein the training data includes at least on of accelerometer data, ambient light data, capacitive touch sensor data, and one or more features of the first intensity and the second intensity.

30-32. (canceled)

33. The device of claim 5, wherein the device is configured to, in response to determining that the physiological monitor is positioned for use on a skin of a user, provide a notification to the user that the device is ready for use.
